(19) United States
(12) Patent Application Publication
(10) Pub. No.: US 2011/0092774 A1
(43) Pub. Date: Apr. 21, 2011

(54) Laryngoscope blade

(76) Inventor: Dwight Herman, Denison, TX (US)

(21) Appl. No.: 12/807,590

(22) Filed: Sep. 9, 2010

Related U.S. Application Data
(60) Provisional application No. 61/276,195, filed on Sep. 9, 2009.

(51) Int. Cl. A61B 1/267

(52) U.S. Cl. 600/190

(57) ABSTRACT

An improved blade for a laryngoscope having a handle, the blade having an elongated body having a central axis, and having a distal tip symmetric about the central axis and having any one of a variety of shapes, including having outer rounded lobes, being generally rectangular, or having a spoon shape; a downwardly convex arcuate central portion including a first flange and a second flange. Each flange has a distal flange tip and a proximal base, the first flange tip being a mirror image of the second flange about the central axis. The outer edge of the base of the first flange is convex and the outer edge of the base of the second flange is concave with respect to the central axis. A light attachment area is provided along the central axis with a bifurcated light. The improvement provides a pair of support structures extending coaxially on either side of a central groove.

### CROSS-REFERENCE TO RELATED APPLICATIONS

This application claims priority from co-pending U.S. Provisional Application Ser. No. 61/276,195 filed Sep. 9, 2009.

### BACKGROUND OF THE INVENTION

1. Field of the Invention

This invention relates to laryngoscopes, and in particular, relates to laryngoscope blades.

2. Description of the Related Art

Laryngoscopes are used to establish an artificial airway in a respiratory compromised person by exposing the glottic opening through displacing the tongue and orpharyngeal tissue, illuminating of the laryngeal opening, and providing tongue and epiglottis stability. Thus, a laryngoscope allows examination of the larynx and aids in endotracheal intubation, such as during surgery or to assist patients to breathe in emergency situations. During intubation, a flexible tube is inserted over the tongue, through the larynx past the vocal cords and epiglottis. It is important in use of a laryngoscope that the blade be structured to keep the tongue and epiglottis from occluding the view of the vocal cords without harming the patient's delicate soft tissue.

Two commonly used laryngoscope blades are the Miller blade and the Macintosh blade. The Miller blade is a relatively narrow straight blade with a slightly elevated tip. This blade sweeps the tongue to the side and lifts the epiglottis directly to allow visualization of the vocal cords so that the tube may be inserted correctly. The Macintosh blade is a wider curved blade, and is used by placing the tip between the epiglottis and the base of the tongue (valecula) and placing pressure to raise the epiglottis enough so that the vocal cords may be viewed.

The structure of these and other prior blades often makes it difficult to see down the patient's throat, due to portions of the structure, such as the light source or blade tip, blocking the area that the practitioner is trying to observe or blocking view of the passageway. Some prior structures often do not sufficiently displace the tongue, epiglottis, and oropharynx for optimal use.

In co-pending U.S. patent application Ser. No. 11/397,835 is disclosed a new laryngoscope blade that features a new curved form with bilateral flanges so that it better displaces oropharyngeal soft tissue and gives a larger, more direct view of the glottic opening, where there is an inability to visualize certain pharyngeal structures. The prior laryngoscope blade of the invention also provides better stability of the tongue and epiglottis during use. Under certain circumstances, however, this prior laryngoscope blade tends to bend if substantial force is used on the laryngoscope blade to lift the jaw to acquire a satisfactory direct view of the laryngeal opening for placement of an endotracheal tube.

It is therefore an object of the invention herein to provide an improved laryngoscope blade that features support structures to stabilize and strengthen it.

Other objects and advantages will be more fully apparent from the following disclosure and appended claims.

### SUMMARY OF THE INVENTION

The invention herein is an improved blade for a laryngoscope having a handle, the blade having an elongated body having a central axis, and having a distal tip symmetric about the central axis; a downwardly convex arcuate central portion including a first flange and a second flange. Each flange has a distal flange tip and a proximal base, the first flange tip being a mirror image of the second flange about the central axis. The outer edge of the base of the first flange is convex and the outer edge of the base of the second flange is concave with respect to the central axis. A light attachment area is provided along the central axis. The improvement in the blade comprises a pair of support structures extending coaxially on either side of a central groove.

Other objects and features of the inventions will be more fully apparent from the following disclosure and appended claims.

### BRIEF DESCRIPTION OF THE DRAWINGS

FIG. 1 is a top perspective view of a first embodiment of the laryngoscope blade of the prior art invention.

FIG. 2 is a top plan view of a second embodiment of the laryngoscope blade of the prior art invention.

FIG. 3 is a top plan view of the distal end of a third embodiment of the laryngoscope blade of the prior art invention.

FIG. 4A is a top plan view of a first embodiment of the support structures of the first embodiment of the laryngoscope blade of the instant invention. FIG. 4B is a top plan view of a second embodiment of the support structures of the first embodiment of the laryngoscope blade of the instant invention.

FIG. 5A is a top plan view of a first embodiment of the support structures of the second embodiment of the laryngoscope blade of the instant invention. FIG. 5B is a top plan view of a second embodiment of the support structures of the second embodiment of the laryngoscope blade of the instant invention.

FIG. 6 is a left side perspective view of the first embodiment of the laryngoscope blade of the prior art invention.

FIG. 7 is a right side perspective view of the first embodiment of the laryngoscope blade of the prior art invention which is cut away as compared to the left side (FIG. 6).

FIG. 8 is a cross-sectional view of FIG. 6 at 8-8 (prior art invention).

FIG. 9 is a cross-sectional view of FIG. 6 at 9-9 (prior art invention).

FIG. 10 is a distal end perspective view of the first embodiment (prior art invention).

FIG. 11A is a left side perspective view of the second embodiment of the laryngoscope blade of the invention having the first embodiment of the support structure of the invention herein. FIG. 11B is a left side perspective view of the second embodiment of the laryngoscope blade of the invention having the second embodiment of the support structure of the invention herein.

FIG. 12A is a right side perspective view of the second embodiment of the laryngoscope blade of the invention which is cut away as compared to the left side (FIG. 11A). FIG. 12B is a right side perspective view of the second embodiment of the laryngoscope blade of the invention which is cut away as compared to the left side (FIG. 11B).

FIG. 13A is a distal end perspective view of the embodiment of FIG. 11A. FIG. 13B is a distal end perspective view of the embodiment of FIG. 11B.

FIG. 14A is a distal end perspective view of the third embodiment of the prior invention showing the first embodiment of the support structure herein. FIG. 14B is a distal end perspective view of the third embodiment of the prior invention showing the second embodiment of the support structure herein.

FIG. 15 is a perspective view of use of the blade of the invention.

FIG. 16 is a partial end view of a laryngoscope blade of the invention showing the first support structure.

FIG. 16A is a partial end view of a laryngoscope blade showing a U-shaped trough A1.

FIG. 17 is a partial end view of a laryngoscope blade of the invention showing the second support structure.

FIG. 17A is a partial end view of a laryngoscope blade showing a V-shaped trough B1.

FIG. 18 is a top plan view of the laryngoscope blade of the invention herein having a bifurcated light fixture.

### DETAILED DESCRIPTION OF THE INVENTION AND PREFERRED EMBODIMENTS THEREOF

The present invention is an improved laryngoscope blade having a flanged structure that compresses the tongue and oropharynx anteriorly as well as displacing the bulk of the tongue distally left and right with the tongue going equally both ways at the same time. In the discussion below, the prior invention of the inventor herein is set forth so that the improvements of the invention herein can be understood in context.

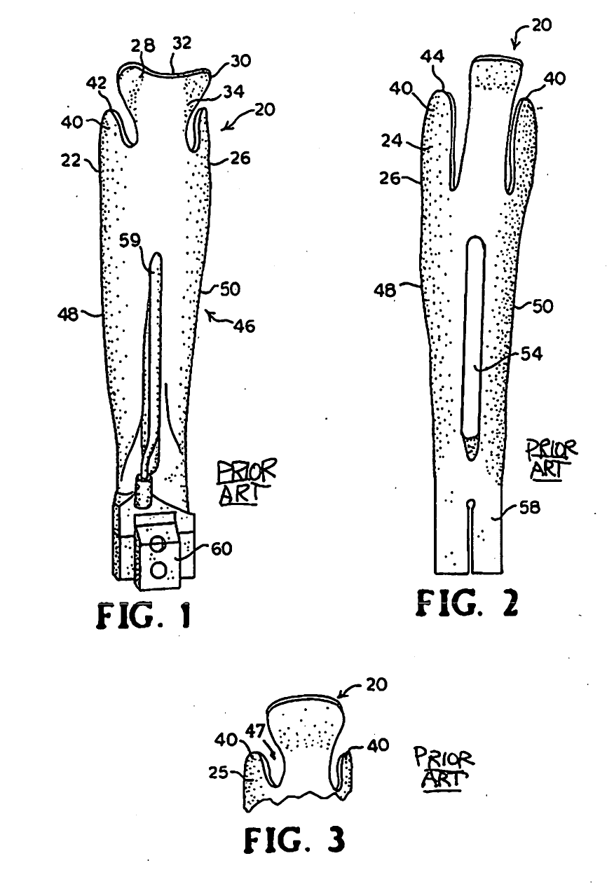

The following discussion is of the prior invention of Ser. No. 11/397,835 as modified and improved herein. The prior invention is shown in FIGS. 1-3 and 6-10. The first embodiment 22 of the laryngoscope blade with a straight tip and a third embodiment 25 with a spoon tip are particularly useful for direct manipulation of the epiglottis, while a second embodiment 24 with a curved tip is particularly useful for manipulating the epiglottis via the valecula.

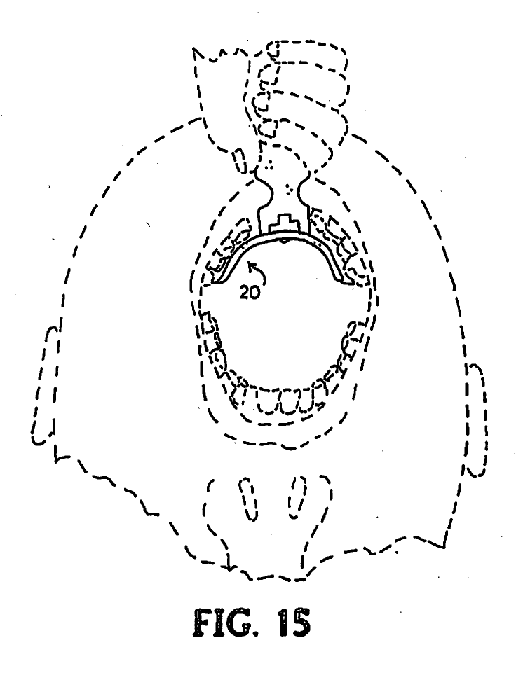

As used herein, the term “downward” or “below” refer to the side of the invention that is inferior (roof of mouth) when the blade is inserted into the throat of a supine person, as shown in FIG. 15. The term “distal” refers to portions of the blade away from the handle, and the term “proximal” refers to portions of the blade closer to the handle. The term “coaxial” means parallel to the central axis which extends from the center of the tip to the center of the handle end.

The laryngoscope blade 20 of the invention may be made of different materials, and is preferably made out of stainless steel, plastic (disposable), or other metals that may be formed into the structure disclosed herein and may be safely used in the mouth. It may be constructed in different sizes and dimensions to accommodate adult and pediatric airways. In addition, the blade 20 may be configured with different light sources (conventional, fiberoptic, and the like) attached to the blade as in the same way as is known in the art, with or without suction apparatus, with or without a tongue gripping apparatus and with or without an epiglottal stabilizing design.

The laryngoscope blade 20 of the invention has three different preferred embodiments of the blade tip: relatively straight tip of the first embodiment (FIGS. 1, 4, 6 and 7), curved tip (as viewed from the side) of the second embodiment (FIGS. 2, 5, 11 and 12), and spoon tip of the third embodiment (FIG. 3)(which would this look like the first embodiment from the side).

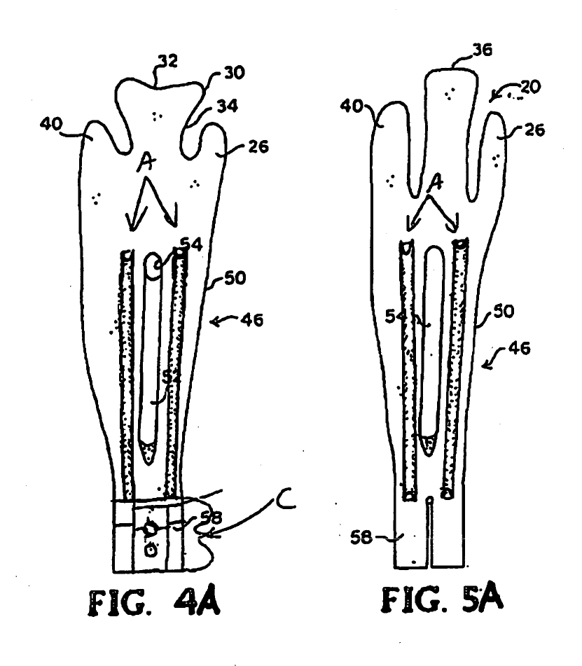

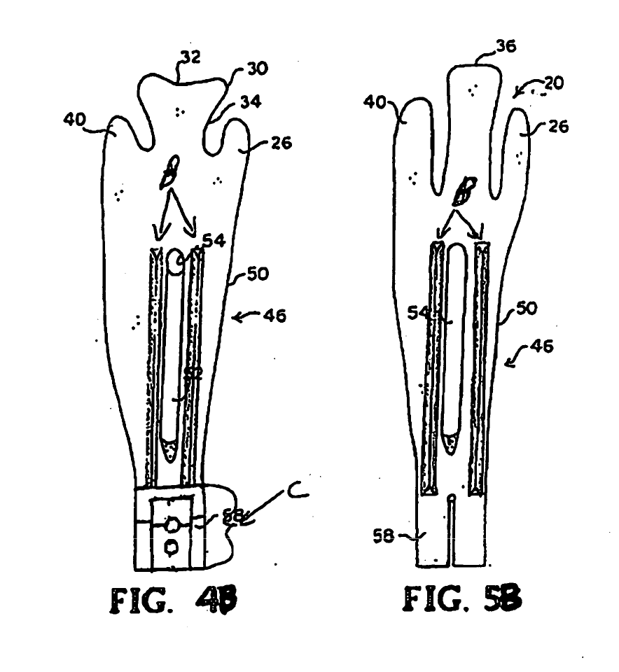

When made of stainless steel, the laryngoscope blade 20 is cut out of a flat piece (preferably less than about 2 mm thick) of stainless steel in the shape shown in FIGS. 4A and 4B for the first embodiment 22 of the laryngoscope blade, and in FIGS. 5A and 5B for the second embodiment 24 of the laryngoscope blade. These figures also show the support structures A and B of the invention herein, which are discussed in more detail below. The blade is preferably about 153 mm long for an average to large woman and a small to large man, but it could be possibly 3 cm shorter or longer without affecting performance, since the width is more critical than the length of the blade. The body of the blade in this embodiment at its widest point 26 is preferably about 50 mm wide, and at its handle end is about 21 mm, to allow attachment to a standard laryngoscope blade handle.

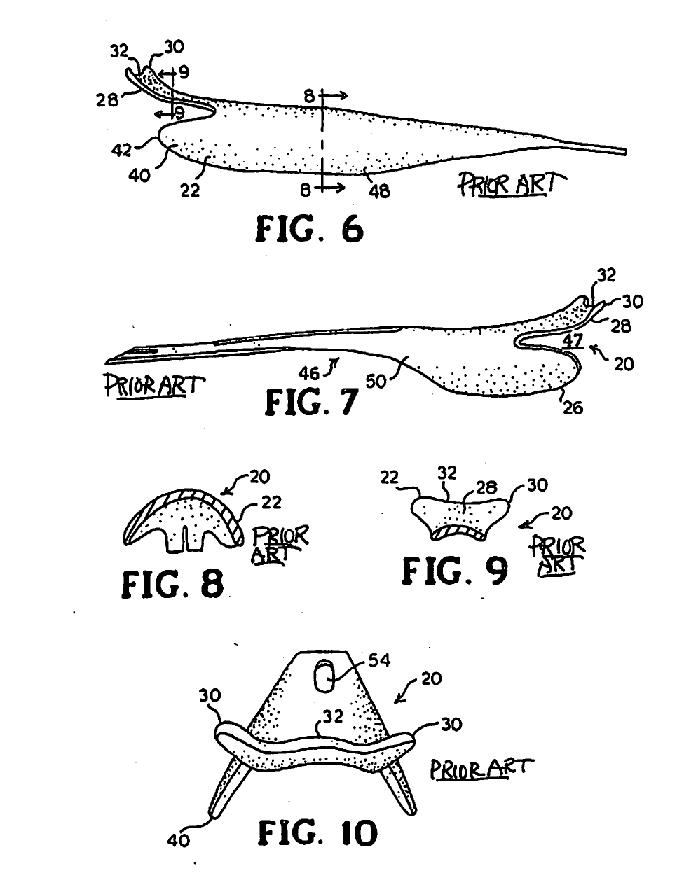

In the first embodiment 22 of the prior laryngoscope blade, the tip 28 is slightly curved upward at the tip when viewed from the side (FIGS. 6-7). When viewed from above or below, the end of the tip 28 has two outer rounded lobes 30 and has a central inward concavity 32 (FIGS. 1 and 4A and 4B). In any large adolescent to adult, the blade tip will accommodate the largest epiglottis (as a large male), and in doing so will also accommodate a smaller epiglottis (as a small adolescent or female). The width at the widest point of the blade should be no smaller than 36-42 mm. The length of the tip is preferably 28-32 mm, and at its narrowest point 34 this tip is preferably about 15.5 mm. This tip is particularly useful for manipulating the epiglottis. In use, viewing the epiglottis as the blade is inserted into the oropharynx, the tip scoops gently under the epiglottis and is lifted anteriorly, exposing the glottic opening so the endotracheal tube can pass via the opening

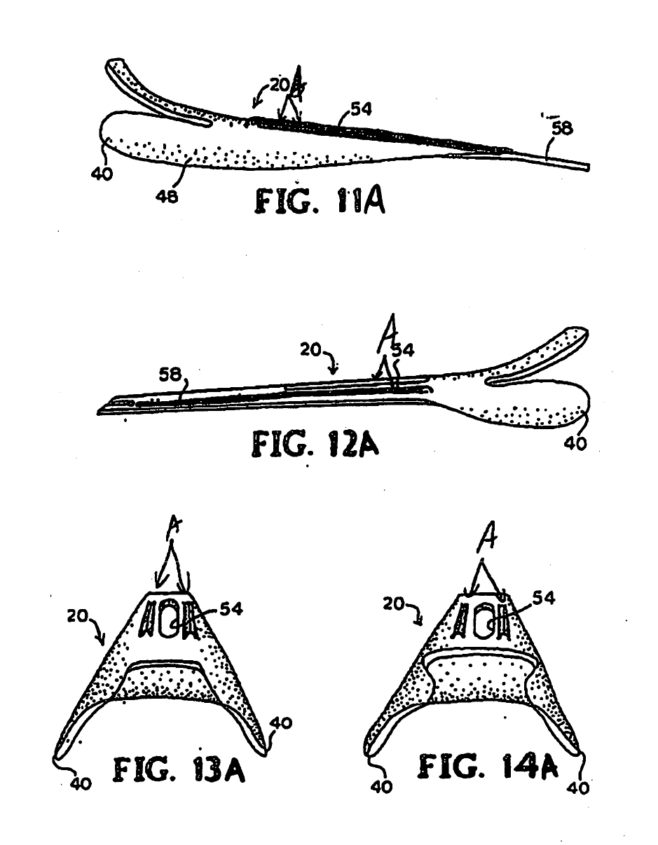

In the second embodiment 24 of the prior laryngoscope blade, the tip 36 is curved when viewed from the side (FIGS. 11A,11B, 12A,12B). These figures also show the support structures A and B of the invention herein, which are discussed in more detail below. When viewed from above or below (FIG. 2 for view from above), the end of the tip 36 in this embodiment is straight as shown, and the tip itself is generally in the form of an elongated rectangle. Preferably the tip of this blade is about 17 mm wide and 40 mm long. This tip is particularly useful for manipulating the valecula. In use, the epiglottis is visualized as the blade is inserted into the oropharynx. The tip is placed in the valecula (tissue area directly in front of the epiglottis) and is lifted anteriorly, causing the epiglottis to flip up (forward and anterior), exposing the glottic opening behind the epiglottis.

In both primary embodiments of the prior laryngoscope blade, the blade has two flanges 40 to displace pharyngeal soft tissue. For the first (straight) embodiment 22. (FIG. 1) for use in a person having a large throat, the distal end 42 of each of these flanges 40 is the same size, which is preferably about 15 mm long and 11 mm wide. The distal ends 42 of the flanges 40 in this embodiment are preferably separated from the base of the tip by a gap of about 6 mm. For the curved tip second embodiment 24, the distal end 44 of each of the flanges 40 is preferably about 30 mm long and about 8-9 mm wide (FIG. 2).

As can be seen in FIGS. 1-2, the blade is not symmetric about its central axis, but rather has an open area 46 on the right side as viewed from above to allow easier viewing past the blade 20 when the blade is being used. This open area 46 can be more easily seen by comparing FIG. 6 with FIG. 7. This open area 46 allows the endotracheal tube to be placed with minimal visual obstruction. Thus, the base 48 of the left flange of each embodiment, as viewed in FIGS. 1 and 2, is convex outward as shown, while the base 50 of the right flange is concave inward. This configuration is preferred for a right-handed person, who typically uses the laryngoscope blade with the left hand, and inserts the endotracheal tube with the right hand; however, the mirror-image of the invention could be made without departing from the spirit and scope of the invention herein. Alternatively, both sides could be cut out, but this configuration would not be as useful in displacing soft tissue during use of the laryngoscope blade.

A press rig or other device as is known in the art is used to attain a uniform curve to the blade. As shown in FIG. 8, the central area of the laryngoscope blade of the invention has a cross-section in the form of an arc, which due to the difference in flange base size, has differently sized arms. A cross-section taken closer to the tip of the flanges (FIG. 9) is shorter with same-length arms and less curvature than the central cross-section shows.

An end view of the distal tip of the three embodiments is shown in FIGS. 10, 13(A&B) and 14(A&B), respectively. In the first embodiment (FIG. 10), the outer rounded points of the blade tip are elevated as shown. The distal ends 42 of the flanges 40 could be thickened and rounded using a soldering or welding technique to increase the surface area, and to decrease trauma to the soft oropharyngeal tissue.

The light source may be attached to the top of the blade in a variety of ways. The prior laryngoscope blade has the light source run along the top of the blade in a trough before poking through the blade to the bottom of the blade about one-third of the way along the blade from the tip, to illuminate the oropharynx. In one embodiment shown in FIG. 4, a trough (groove) 52 is pressed down the middle of the blade, extending about 70 mm and having a width of about 25 mm. A central hole 54 having a diameter of about 2.5 mm is located at the end of the groove for placement of the light source. Alternatively, a slot 56, 50 mm×2.5 mm, may be formed down the central axis of the blade as shown. In either case, the handle end 58 of the blade extends about 30-35 mm from the proximal end of the slot or trough.

The preferred embodiment of the invention herein, however, has a bifurcated light source LL as shown in FIG. 18. This feature is added to the blade to increase the efficacy, brightness, and intensity of the light source is a bifurcation in the light source LL, as shown in FIG. 18, which is preferably at 3-7 cm from proximal end of the light source. The bilateral length of the bifurcation branches from the point of the fork is preferably 2-6 cm long. The light source can be any current lighting technology (standard, fiberoptic, halogen, etc. as known in the art). The light source runs along top of blade with the distal tips protruding through the blade so as to shine light on the underside of the blade. The distal tips of the light source curve inward so as to blend the bilateral light beam to an optimal light source for direct visualization of the oropharynx. The bilateral light source tips are preferably 2-4 cm's apart from each other at most distal end.

The light source 59 (shown in FIG. 1), preferably a standard fiberoptic light source as is known in the art, is attached to the blade 20 by welding or soldering as is known in the art.

A standard handle connection fixture 60 is preferably attached to the handle end 58 of the blade 20 of the invention so that the laryngoscope blade of the invention may be used with standard handles known in the art, and the handle end 58 as shown in the figures is configured to attach thereto.

Optionally, a suction source, e.g., a metal tube, may be placed along the larger flange directing a disposable suction catheter toward the posterior oropharynx (not shown).

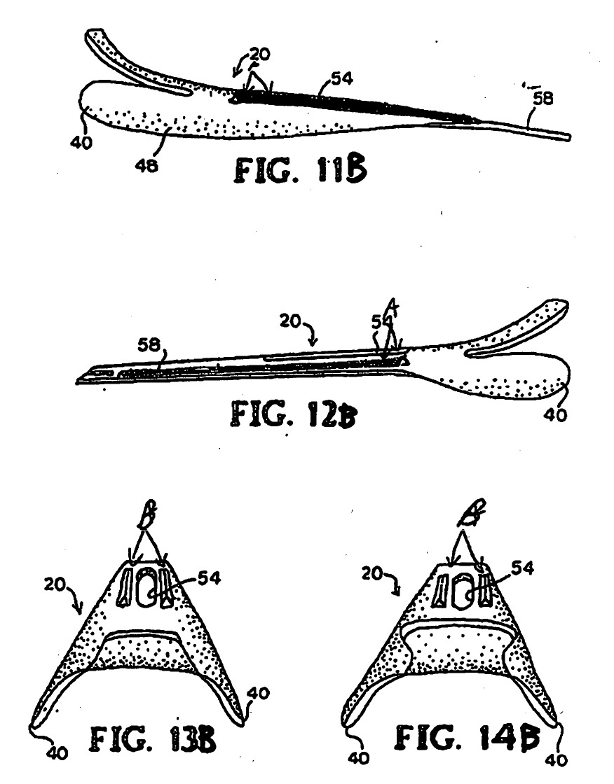

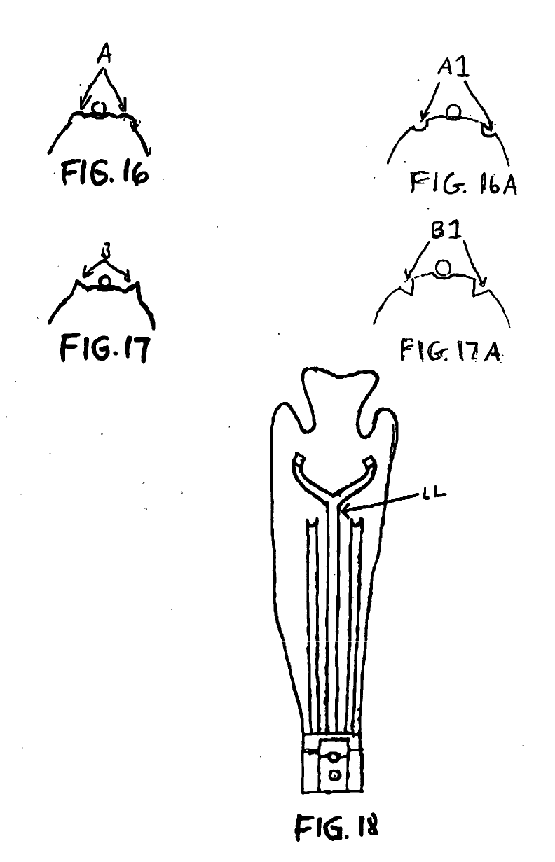

The prior laryngoscope blade tends to bend at the proximal end of the blade causing the material to bend under the force that is used to lift the jaw to acquire a satisfactory direct view of the laryngeal opening for placement of an endotracheal tube. In the invention disclosed herein there are two embodiments of support structures, termed support structure A (FIGS. 4A, 5A, 11A, 12A, 13A, 14A and 16) and support structure B (FIGS. 4B, 5B, 11B, 12B, 13B, 14B and 17), which extend coaxially along the central groove. These support structures solve this shortcoming. This is done by creating a peak that is rounded (support structure A) or pointed (support structure B) or trough that is rounded (support structure Al1in FIG. 16A) or pointed (support structure B1 in FIG. 17A) by adding material adhering plastic by gluing or any other adhesive process, or metal by welding or any other adhesive process in the shape of a U, inverse U, V or inverse V to the blade surface or stamping out the shape of a U, inverse U, V or inverse V in the existing material either plastic or metal, preferably using a peak in the material bilaterally on either side of the light source running horizontally preferably 2-6 mm from and alongside the light source. These structures run approximately ½ to ⅔ the length of the blade from proximal to distal, and thus are preferably at least 2-8 cm long and are preferably about 2-5 mm wide and are preferably about 2-5 mm deep. The proximal end of these structures would preferably, but not necessarily, be attached to an attachment portion 60 (structure C on FIGS. 4A and 4B) at the proximal end of the blade giving more rigidity to the blade. The attachment portion 60 comprises of a machined material either plastic or metal that exists on proximal end of all current standard laryngoscope blades, which is used to attach said laryngoscope blade to a battery light source handle. This attachment apparatus is usually welded in the case of a metal blade or molded along with the blade portion of a plastic laryngoscope blade. The means of attachment of the support structures to the attachment portion is by any welding process with the metal blade and molding to the attachment portion in a plastic version of the blade. The structures can be either U shaped or inverse U shaped as in structure A in FIGS. 4A, 5A, 11A, 12A, 13A, 14A and 16, and structure A1 as in FIG. 16A, as well as V or inverse V shaped as in structures B in FIGS. 4B, 5B, 11B, 12B, 13B, 14B and 17, and structure B1 in FIG. 17A. This U or V shape is formed by molding or stamping the material. Other comparably constituted support structures, including bending the metal of the proximal end of the blade in any way as known in the art, or adding metal support structures design that solves the shortcoming stated above is also included in the patent.

All the measurements set forth herein could vary based on different sizing of the blade as known in the art.

While the invention has been described with reference to specific embodiments, it will be appreciated that numerous variations, modifications, and embodiments are possible, and accordingly, all such variations, modifications, and embodiments are to be regarded as being within the spirit and scope of the invention.

What is claimed is:

1. A blade for a laryngoscope having a handle, the blade comprising an elongated body having a central axis, and having:
a) a distal tip symmetric about the central axis;
b) a downwardly convex arcuate central portion comprising:
i) a first flange and a second flange, each flange having a distal flange tip and a proximal base, the first flange tip being a mirror image about the central axis of the second flange tip; the outer edge of the base of the first flange being convex and the outer edge of the base of the second flange concave with respect to the central axis; and
ii) a light attachment area along the central axis;
c) a proximal handle attachment portion; and
d) a pair of support structures extending coaxially on either side of a central groove.

2. The blade for a laryngoscope of claim 1, wherein each support structure comprises a rounded peak.

3. The blade for a laryngoscope of claim 1, wherein each support structure compriks a pointed peak.

4. The blade for a laryngoscope of claim 1, wherein each support structure comprises a rounded trough.

5. The blade for a laryngoscope of claim 1, wherein each support structure comprises a pointed peak.

6. The blade for a laryngoscope of claim 1, further comprising a bifurcated light source.

7. The blade for a laryngoscope of claim 1, wherein the support structures have a proximal end that is attached to the proximal handle attachment portion.
